(19) United States
(12) Patent Application Publication
(10) Pub. No.: US 2024/0400999 A1
(43) Pub. Date: Dec. 5, 2024

(54) EXTRACELLULAR MATRIX FOR THREE-DIMENSIONAL CELL CULTURE AND METHOD FOR PRODUCING SAME

(71) Applicant: ORG CORP., Goyang-si, Gyeonggi-do (KR)

(72) Inventor: Jaeyeaon CHO, Goyang-si Gyeonggi-do (KR)

(73) Assignee: ORG CORP., Goyang-si, Gyeonggi-do (KR)

(21) Appl. No.: 18/705,405

(22) PCT Filed: Oct. 28, 2021
(86) PCT No.: PCT/KR2021/015321

§ 371 Date: Apr. 26, 2024

(30) Foreign Application Priority Data
Oct. 27, 2021 (KR) 10-2021-0145017

(51) Int. Cl. C12N 5/071; C12M 1/12; C12N 5/00; C12N 5/077; C12N 9/76

(52) U.S. Cl. CPC C12N 5/0697; C12M 25/14; C12N 5/0062; C12N 5/0656; C12N 9/6427; C12N 2500/38; C12N 2533/52; C12Y 304/21004

(57) ABSTRACT

Provided in the present specification are a method for producing an extracellular matrix, the method comprising the steps of: culturing fibroblasts in a stimulation medium so as to form a fibroblast patch comprising the fibroblasts and an extracellular matrix; treating the formed fibroblast patch with a proteinase; freezing the proteinase-treated fibroblast patch; thawing the frozen fibroblast patch so that the fibroblasts are decellularized from the fibroblast patch; and obtaining the extracellular matrix from the decellularized fibroblast patch, and an extracellular matrix produced on the basis of the method, the extracellular matrix comprising collagen, actinin, and an actin-binding-like protein (filamin-C) and being present in the form of entangled nanofibers.

### CROSS REFERENCE TO RELATED APPLICATION(S)

This application is a 35 U.S.C. § 371 National Phase Entry Application from PCT/KR2021/015321, filed on Oct. 28, 2021, which claims the benefit of Korean Patent Application No. 10-2021-0145017, filed on Oct. 27, 2021, the disclosures of which are herein incorporated by reference in their entirety.

### TECHNICAL FIELD

The present invention relates to an extracellular matrix for three-dimensional cell culture and a method for producing the same.

### BACKGROUND ART

Organoids have attracted attention as a new biomimetic model. The organoids are formed into three-dimensional structures such as organs by growing stem cells into specific cells. Unlike the two-dimensional cell-based model, the organoids may be cultured in a three-dimensional environment and cultured for a longer period of time. In addition, the organoids are small in size, but constituent cells and structures thereof are similar to those of a real organ. Accordingly, the organoids have been evaluated as optimal specimens for examining the efficacy and stability of drugs in the process of developing new drugs. Furthermore, organoid-related fields have high potential to be used not only for evaluating the drug toxicity and efficacy of new drug development, but also for disease models, cancer research, personalized medicine, regenerative therapeutics, and the like.

In order to produce such organoids, an ultrastructural scaffold is important as an environmental element for specific cells to grow, proliferate, and form into target organoids. More specifically, for survival, cells require transport of materials through the cell membrane and signaling through membrane proteins present in a suspension state in the cell membrane and a microstructure of the scaffold surface may control the adsorption, proliferation, and differentiation characteristics of cells by modifying the cell membrane into a desired shape.

Accordingly, in order to differentiate cells into desired target organoids, a scaffold similar to an in vivo environment is required, and an extracellular matrix (ECM) has been currently used as such a scaffold.

The extracellular matrix consists of proteins and polysaccharides secreted by cells. The extracellular matrix has a specific composition for each tissue (organ) and contains growth factors secreted by cells, so as to have a comprehensive effect on adhesion, proliferation, migration, and differentiation of cells. Conventionally, the extracellular matrix has been used by decellularizing organs or tissues obtained directly from a subject. However, in this method, since the decellularization process varies depending on an organ or tissue and an actual organ of a subject has a structure that is mixed with not only a target tissue, but also other tissues such as fat and blood vessels, in order to completely remove other tissues and obtain only the target extracellular matrix, various treatments such as acids, bases, enzymes, surfactants, and heat are performed, and thus the extracellular matrix may be modified.

Furthermore, since the modified extracellular matrix is different from a tissue environment of an original subject, differentiation into a desired tissue may not occur (low tissue differentiation), thereby deteriorating the uniformity and reproducibility of organoid production. In addition, a conventional extracellular matrix has a limit to the supply and demand of subjects, and limitations that cost and time requirements are considerable, by using tissues obtained from the subjects.

For this reason, there have been attempts to use the extracellular matrix secreted after adherent culture of cells as a scaffold for cell culture. For example, in order to use a network structure of the extracellular matrix as it is (Korean Patent Publication No. 1020140136975) or to preserve the instability of the structure, there has been provided a method using a poly(octadecene-alt-maleic anhydride) system for inducing covalent bonds with molecules having amino terminal groups. However, the method is a method using spin coating, which requires the provision of equipment for spin coating and has the inconvenience of further performing a process of coating fibronectin and the like, and has a disadvantage of a short preservation period of about 4 weeks, and the like.

The background art of the invention has been prepared to more facilitate understanding of the present invention. It should not be understood that the matters described in the background art of the invention exist as prior arts.

### DISCLOSURE

### Technical Problem

Meanwhile, the present inventors recognized that conventional three-dimensional cell culture using an extracellular matrix had structural limitations. More specifically, the present inventors recognized that conventional three-dimensional culture was performed by coating an extracellular matrix on a plate with a limited thickness of 10 to 15 μm to provide no sufficient spatial environment where cells grew into tissues.

Furthermore, the present inventors found that in the process of forming the extracellular matrix, when adding ascorbic acid at a specific concentration, a fibroblast-derived extracellular matrix may be formed even without adding a protein element such as a growth factor. In other words, since the extracellular matrix may be produced only with low-cost chemical elements rather than high-cost protein elements such as growth factors, it is possible to produce the extracellular matrix with high efficiency.

Ultimately, the present inventors have developed an extracellular matrix having structural elements capable of causing a limited growth, that is, sufficient thickness and size and a shape similar to a structure in vivo, and a production method with high efficiency capable of reducing production cost thereof.

Therefore, an object of the present invention is to provide an extracellular matrix overcoming structural limitations, including collagen, actinin, and an actin-binding-like protein (filamin-C), and being present in the form of entangled nanofibers, and a method for producing the extracellular matrix.

The objects of the present invention are not limited to the aforementioned objects, and other objects, which are not mentioned above, will be apparent to those skilled in the art from the following description.

### Technical Solution

According to an exemplary embodiment of the present invention, there is provided a method for producing an extracellular matrix including the steps of: culturing fibroblasts in a stimulation medium so as to form a fibroblast patch including the fibroblasts and an extracellular matrix; treating the formed fibroblast patch with a proteinase; freezing the proteinase-treated fibroblast patch; thawing the frozen fibroblast patch so that the fibroblasts are decellularized from the fibroblast patch; and obtaining the extracellular matrix from the decellularized fibroblast patch.

According to the feature of the present invention, the stimulation medium may include 0.01 to 2 mM ascorbic acid, but is not limited thereto.

According to another feature of the present invention, the culturing step may be performed for at least one period of 3 to 20 weeks, but is not limited thereto. According to yet another feature of the present invention, the proteinase may be 0.01 to 1% trypsin, but is not limited thereto.

According to yet another feature of the present invention, the freezing step may be performed at a temperature of −10° C. or lower but is not limited thereto.

According to yet another feature of the present invention, the thawing step may be performed at room temperature for at least 2 hours, but is not limited thereto.

According to yet another feature of the present invention, the method may further include treating the thawed fibroblast patch with a proteinase, after the thawing step.

According to yet another feature of the present invention, the method may further include freeze-drying the obtained extracellular matrix, after the obtaining step.

According to yet another feature of the present invention, the extracellular matrix obtained in the obtaining step may include fibronectin.

According to yet another feature of the present invention, the fibroblasts may be derived from at least one of tendon, ligament, muscle, skin, periodontium, cornea, cartilage, bone, liver, blood vessels, heart, small intestine, large intestine, and intervertebral disc, but are not limited thereto.

According to another exemplary embodiment of the present invention, there is provided an extracellular matrix including collagen, actinin, and an actin-binding-like protein (filamin-C), and being present in the form of entangled nanofibers.

According to the feature of the present invention, the nanofibers may have a thickness of about 0.5 μm or less, but are not limited thereto.

According to another feature of the present invention, the extracellular matrix may have elasticity and compliance when including cells, and have no elasticity and compliance when not including the cells.

According to yet another feature of the present invention, the extracellular matrix may allow the culture medium to be movable between the entangled nanofibers.

According to yet another feature of the present invention, the extracellular matrix may include at least one of ACTN1, ACTN4, ANXA2, ANXA4, ANXA5, ANXA6, CAV1, COL1A1, COL1A2, COL3A1, COL5A2, COL6A1, COL6A2, COL6A3, FLNC, GPNMB, GSTM3, LMNA, LUM, MYH9, PLEC, PRDX1, PRDX4, PRELP, PTRF, THY1 and VAT1, but is not limited thereto.

According to yet another feature of the present invention, the extracellular matrix may be derived from fibroblasts in vitro.

According to yet another feature of the present invention, the extracellular matrix may have the same structure as an extracellular matrix in vivo.

According to yet another feature of the present invention, the extracellular matrix may be in a semisolid phase.

According to yet another feature of the present invention, the extracellular matrix may be for three-dimensional cell culture.

According to yet another exemplary embodiment of the present invention, there is provided a composition for three-dimensional cell culture, including the extracellular matrix described above.

At this time, the extracellular matrix may have a chopped form, but is not limited thereto.

According to yet another exemplary embodiment of the present invention, there is provided a dish for three-dimensional cell culture, including the extracellular matrix described above.

At this time, the extracellular matrix may have a dried form, but is not limited thereto.

Hereinafter, the present invention will be described in more detail with reference to exemplary embodiments. However, these exemplary embodiments are only illustrative the present invention, and the scope of the present invention is not limited to these exemplary embodiments.

### Advantageous Effects

According to the present invention, it is possible to provide a culture environment capable of properly differentiating specific cells into target organoids. More specifically, since an extracellular matrix for three-dimensional cell culture according to an exemplary embodiment of the present invention has improved thickness and size compared to a conventional extracellular matrix with limited thickness (50 μm) and size, it is possible to provide a more improved three-dimensional culture environment for the formation of organoids.

In other words, the present invention has an effect of providing a culture environment most similar to an in vivo environment.

Furthermore, an extracellular matrix for four-dimensional cell culture according to an exemplary embodiment of the present invention does not require administration of growth factors consisting of proteins, etc., and may be produced only with cheap chemicals, thereby providing a high-efficiency scaffold at low cost and being more economical.

The effects according to the present invention are not limited to the contents exemplified above, and further various effects are included in the present specification.

### DESCRIPTION OF DRAWINGS

FIGS. 1A and 1B exemplarily illustrate a procedure of a method for producing an extracellular matrix according to an exemplary embodiment of the present invention.

FIG. 2 illustrates genetic analysis results for an extracellular matrix according to an exemplary embodiment of the present invention.

FIGS. 3A to 3C illustrate observation image results of an extracellular matrix according to an exemplary embodiment of the present invention produced through the above-described process.

FIG. 4 illustrates electron microscopy image results for an extracellular matrix according to an exemplary embodiment of the present invention.

FIG. 5 illustrates observation image results for phase changes of an extracellular matrix according to an exemplary embodiment of the present invention.

FIG. 6 is an exemplary diagram of a cell culture method of an extracellular matrix according to an exemplary embodiment of the present invention.

FIGS. 7A and 7B are exemplary diagrams of a composition for three-dimensional cell culture and a dish for three-dimensional cell culture including an extracellular matrix according to an exemplary embodiment of the present invention.

FIGS. 8A and 8B are cell culture results in an extracellular matrix according to an exemplary embodiment of the present invention.

### BEST MODE OF THE INVENTION

Advantages and features of the present invention, and methods for accomplishing the same will be more clearly understood from exemplary embodiments to be described below in detail with reference to the accompanying drawings. However, the present invention is not limited to the exemplary embodiments set forth below, and will be embodied in various different forms. The exemplary embodiments are just for rendering the disclosure of the present invention complete and are set forth to provide a complete understanding of the scope of the invention to those skilled in the art to which the present invention pertains.

As used in the present specification, the term “pluripotent stem cells” may refer to cells having the ability to differentiate into all cells constituting the body, and generally, may include induced pluripotent stem cells (iPSCs) and embryonic stem cells (ES cells) having a common characteristic of differentiating to pluripotency. More specifically, the embryonic stem cells are induced from the inner cell mass of blastocysts in a pre-implantation stage. The induced cells are maintained in a specific environment and are capable of unrestricted culture and pluripotent differentiation. Furthermore, the induced pluripotent stem cells may refer to pluripotent differentiated cells formed by dedifferentiation from body somatic cells, and are formed by making somatic cells into a phase very similar to embryonic stem cells through a process called reprogramming, such as cell fusion, nuclear substitution, and overexpression of pluripotency regulators. Furthermore, the pluripotent stem cells are not limited to the embryonic stem cells and the induced pluripotent stem cells, and may include cells having both pluripotent differentiation and self-replication ability. However, preferably, the pluripotent stem cells may be mammalian cells, more preferably human-derived pluripotent stem cells.

As used in the present specification, the term “cardiomyocytes” refer to cells constituting the heart. The cardiomyocytes may be divided into atrial, ventricular and nodal cardiomyocytes according to a structure of the heart, and the cardiomyocytes may be mononuclear cells and may lose a division function in the human body after birth. Therefore, the recovery of damaged cardiomyocytes may be difficult. On the other hand, the cardiomyocytes may be damaged or destroyed when exposed to stress such as cardiac infarction or myocarditis. Accordingly, the damage or destruction of the cardiomyocytes causes a decrease in a myocardial function, which may lead to heart disease. Accordingly, in regenerative cell therapy for recovery of the cardiac function or treatment of heart diseases, cardiomyocytes differentiated from stem cells may be used.

As used in the present specification, the term “differentiation” means that cells are developed at a level of a complex of specific cells or tissues or subject having a specific function.

As used in the present specification, the term “organoid” refers to a small embryoid body that reproduces both the form and the function of a tissue or organ. More specifically, the organoid needs to include one or more cell types among various types of cells constituting the organ or tissue, and needs to be able to reproduce a specific function of each organ, and the cells need to be agglomerated with each other to be spatially organized in a form similar to the organ. The organoid is different from a spheroid in that the organoid forms a lineage rather than a simple aggregate of cells, and may be used for new-drug development, artificial organs, disease therapeutic agents, and patient-specific models for disease treatment.

As used in the present specification, the term “medium” refers to a mixture for the growth and proliferation of cells such as stem cells and the like in vitro, including essential elements for the cell growth and proliferation, such as sugars, amino acids, various nutrients, serum, growth factors, minerals, and the like.

As used in the present specification, the term “proteinase” refers to an enzyme capable of isolating an intercellular matrix in order to liberate cells or cell aggregates included in a living tissue, and may be used with collagenase, dispase, protease, trypsin, etc. to isolate the pluripotent stem cells or to isolate the cells and cell aggregates from the tissue but is not limited thereto.

Furthermore, the term “plate” as used in the present specification is not limited as long as the cell culture may be performed, and may be used with various shaped plates such as flasks, tissue culture flasks, dishes, petri dishes, micro plates, micro well plates, micro slides, Chamber slides, chalets, tubes, trays, and culture bags, and may include a cell adhesion layer coating film on an upper surface thereof.

### Extracellular Matrix According to an Exemplary Embodiment of the Present Invention and Method for Producing the Same

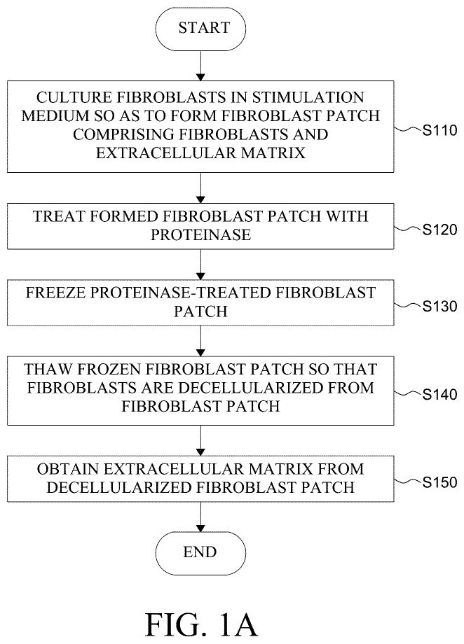

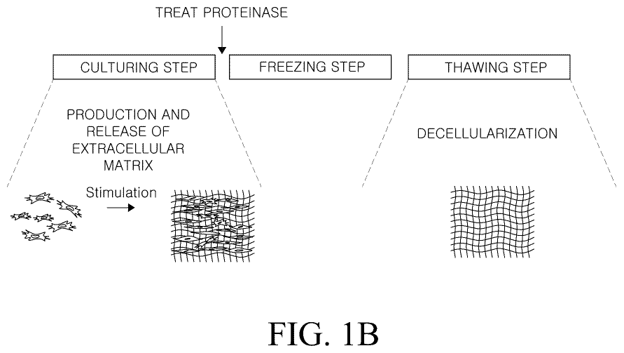

Hereinafter, an extracellular matrix according to an exemplary embodiment of the present invention and a method for producing the same will be described in detail with reference to FIGS. 1A to 5.

FIGS. 1A and 1B exemplarily illustrate a procedure of a method for producing an extracellular matrix according to an exemplary embodiment of the present invention.

First, FIG. 1A is a flowchart of a method for producing an extracellular matrix according to an exemplary embodiment of the present invention, which will be described with reference to FIG. 1B for convenience of explanation.

Referring to FIG. 1A, a method for producing an extracellular matrix according to an exemplary embodiment of the present invention includes the steps of culturing fibroblasts in a stimulation medium so as to form a fibroblast patch including fibroblasts and an extracellular matrix (S110), treating the formed fibroblast patch with a proteinase (S120), freezing the proteinase-treated fibroblast patch (S130), thawing the frozen fibroblast patch so that the fibroblasts are decellularized from the fibroblast patch (S140), and obtaining the extracellular matrix from the decellularized fibroblast patch (S150).

At this time, in the step (S110) of culturing in the stimulation medium, the stimulation medium may include 0.01 to 2 mM ascorbic acid.

More specifically, the ascorbic acid is an antioxidant that is involved in procollagen synthesis and is a cofactor associated with increased type 1 collagen production. The ascorbic acid may stimulate and regulate the proliferation of various cells such as adipocytes, osteoblasts, and chondrocytes in vitro. Furthermore, when a certain concentration of ascorbic acid is added, the ascorbic acid acts as a cell growth promoter to increase cell proliferation, and even promote DNA synthesis. However, when the concentration of ascorbic acid is not appropriate, the ascorbic acid may inhibit the proliferation of cells and be cytotoxic to cause apoptosis. Accordingly, the appropriate concentration of ascorbic acid capable of improving the proliferation of cells, that is, the synthesis and excretion of the extracellular matrix, may be 0.01 to 1 mM, but is not limited thereto, and the more preferable concentration of ascorbic acid may be 0.1 to 1 mM.

Furthermore, since the stimulation medium is a medium containing ascorbic acid, the stimulation medium contains a basic medium as a basis. For example, the basic medium is a mixture containing sugars, amino acids, and water required for cells to live, excluding serum, nutrients, and various growth factors. The basic medium of the present invention may be artificially synthesized and used, or a commercially produced medium may be used. For example, commercially prepared media may include Dulbecco's Modified Eagle's Medium (DMEM), DMEM/F-12, Minimal Essential Medium (MEM), Basal Medium Eagle (BME), RPMI 1640, F-10, F-12, α-Minimal Essential Medium (α-MEM), Glasgow's Minimal Essential Medium (G-MEM), Iscove's Modified Dulbecco's Medium, and Fetal bovine serum (FBS), etc., but are not limited thereto, and preferably DMEM/F-12.

Furthermore, the stimulation medium may further include 0.0001 to 0.001% of acetic acid, but is not limited thereto.

In addition, in the step (S110) of culturing in the stimulation medium, the culture period may be at least one period of 3 to 20 weeks, but is not limited thereto, and preferably at least one period of 6 to 12 weeks.

Accordingly, referring to FIG. 1B, through the above-described culturing step (S110), the fibroblasts are stimulated by ascorbic acid to produce and release the extracellular matrix, and the fibroblast patch including the fibroblasts and the extracellular matrix may be formed.

At this time, the fibroblasts, which produce and release the extracellular matrix, are a type of biological cells that synthesizes the extracellular matrix and collagen and are the most common connective tissue that creates the structural framework in animal tissue and may be obtained from various tissues such as heart tissue. For example, the fibroblasts may be derived from at least one of tendon, ligament, muscle, skin, periodontium, cornea, cartilage, bone, liver, blood vessels, heart, small intestine, large intestine, and intervertebral disc, but are not limited thereto.

Referring back to FIG. 1A, after the culturing step (S110), the decellularization process may be performed to extract only the extracellular matrix from the fibroblast patch, and the decellularization process may include treating the proteinase (S120), freezing the fibroblast patch (S130), and thawing the fibroblast patch (S140). Furthermore, the extracellular matrix to be used for cell culture, that is, as a biomaterial, needs to have as many three-dimensional structures and physiologically active substances of the extracellular matrix as possible in order for specific cells to maintain physiological characteristics. Accordingly, as antigenicity that may cause immune rejection needs to be removed, the decellularization process that removes everything except cells that serve as structures may be essential.

Accordingly, referring to FIG. 1B, the step (S120) of treating the formed fibroblast patch with the proteinase may be performed. At this time, the proteinase may be 0.01 to 1% trypsin, but preferably 0.25% trypsin. However, the proteinase is not limited thereto, and may include all proteinases capable of decomposing binding proteins between the fibroblasts and the extracellular matrix. For example, the proteinase may include Collagenase, Elastase, Dispase, Protease, Pepsin, Rennin, Chymotrypsin, Erepsin, Enterokinase, Peptidase, Proteinase, etc.

Next, the step (S130) of freezing the proteinase-treated fibroblast patch may be performed. At this time, the freezing step may be performed at a temperature of −10° C. or lower but is not limited thereto.

Next, the step (S140) of thawing the frozen fibroblast patch to decellularize the fibroblasts in the fibroblast patch may be performed. At this time, the thawing step may be performed at room temperature for 2 hours or more.

Accordingly, through the decellularization process of S120 to S140 described above, the fibroblasts are released from the fibroblast patch, and only the extracellular matrix without containing fibroblasts and other cells may be obtained (S150).

Meanwhile, the decellularization process may be performed even if the above-described freezing and thawing (S130 and S140) are not included. More specifically, the method for producing the extracellular matrix according to an exemplary embodiment of the present invention may further include treating a decellularization buffer, after the step (S120) of treating the proteinase as the process for decellularization, and at this time, the decellularization buffer may include Triton-X or EDTA. However, the composition of the decellularization buffer is not limited to the above-described Triton-X or EDTA, and may include all nonionic surfactant components commercially used in the art of the present invention.

Furthermore, the method for producing the extracellular matrix according to an exemplary embodiment of the present invention may further include treating the thawed fibroblast patch with a proteinase, when the decellularization is not completely performed, after the thawing step (S140). At this time, in the step of treating the thawed fibroblast patch with the proteinase, the proteinase may be under the same conditions as the proteinase described above, and then the decellularization may be performed through the same process as the above-mentioned process. However, it is not limited thereto, and for example, the step of treating the thawed fibroblast patch with the proteinase may be performed while stirring by adding the proteinase and PBS to the fibroblast patch in a predetermined temperature water bath at 37° C., without performing the freezing and thawing. Furthermore, at this time, the PBS may contain 3% triton-X and 0.05% EDTA, but is not limited thereto.

Furthermore, the method for producing the extracellular matrix according to an exemplary embodiment of the present invention may further include freeze-drying the obtained extracellular matrix, after the obtaining step (S150), but is not limited thereto, and through the freeze-drying step, distribution and supply of the obtained extracellular matrix may be facilitated.

Accordingly, the method for producing the extracellular matrix according to an exemplary embodiment of the present invention may be more economical than a conventional method for producing an extracellular matrix, by producing the extracellular matrix only in a simple process of adding only one type of chemical of ascorbic acid and freezing and thawing.

The extracellular matrix (ECM) according to an exemplary embodiment of the present invention produced through the above process refers to a scaffold in the development of a tissue having a three-dimensional structure which plays an important role in providing signals affecting various cellular metabolic pathways such as proliferation, differentiation and death of the cells. The extracellular matrix may store and supply biochemical factors required for the growth and differentiation of the cells, and provide a physical environment which may be recognized by the cells at the same time. The extracellular matrix is a product produced by cells constituting each tissue as needed, and includes structural proteins such as collagen and elastin, polysaccharides such as glycosaminoglycan (GAG), adhesive proteins that helps the adhesion of cells, and growth factors. The extracellular matrix consists of different components depending on the tissue and cells to be derived, and has special physical properties, and the extracellular matrix according to an exemplary embodiment of the present invention may essentially contain fibronectin.

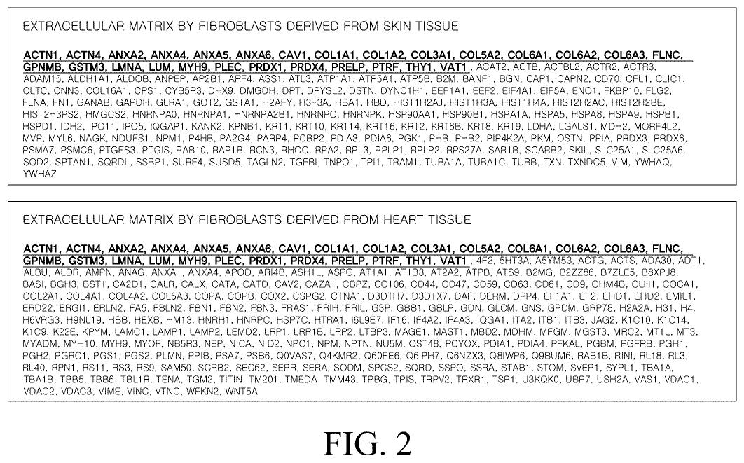

More specifically, referring to FIG. 2, genetic analysis results for the extracellular matrix according to an exemplary embodiment of the present invention are illustrated.

The composition of the extracellular matrix according to an exemplary embodiment of the present invention produced by the above-described process may vary depending on the origin of the fibroblasts. For example, when the fibroblasts are derived from skin (dermis) tissue, the extracellular matrix may include at least one of ACTN1, ACTM4, ANXA2, ANXA4, ANXA5, ANXA6, CAV1, COL1A1, COL1A2, COL3A1, COL5A2, COL6A1, COL6A2, COL6A3, FLNC, GPNMB, GSTM3, LMNA, LUM, MYH9, PLEC, PRDX1, PRDX4, PRELP, PTRF, THY1, VAT1, ACAT2, ACTB, ACTBL2, ACTR2, ACTR3, ADAM15, ALDH1A1, ALDOB, ANPEP, AP2B1, ARF4, ASS1, ATL3, ATP1A1, ATP5A1, ATP5B, B2M, BANF1, BGN, CAP1, CAPN2, CD70, CFL1, CLIC1, CLTC, CNN3, COL16A1, CPS1, CYB5R3, DHX9, DMGDH, DPT, DPYSL2, DSTN, DYNC1H1, EEF1A1, EEF2, EIF4A1, EIF5A, ENO1, FKBP10, FLG2, FLNA, FN1, GANAB, GAPDH, GLRA1, GOT2, GSTA1, H2AFY, H3F3A, HBA1, HBD, HIST1H2AJ, HIST1H3A, HIST1H4A, HIST2H2AC, HIST2H2BE, HIST2H3PS2, HMGCS2, HNRNPAO, HNRNPA1, HNRNPA2B1, HNRNPC, HNRNPK, HSP90AA1, HSP90B1, HSPA1A, HSPA5, HSPA8, HSPA9, HSPB1, HSPD1, IDH2, IPO11, IPO5, IQGAP1, KANK2, KPNB1, KRT1, KRT10, KRT14, KRT16, KRT2, KRT6B, KRT8, KRT9, LDHA, LGALS1, MDH2, MORF4L2, MVP, MYL6, NAGK, NDUFS1, NPM1, P4HB, PA2G4, PARP4, PCBP2, PDIA3, PDIA6, PGK1, PHB, PHB2, PIP4K2A, PKM, OSTN, PPIA, PRDX3, PRDX6, PSMA7, PSMC6, PTGES3, PTGIS, RAB10, RAP1B, RCN3, RHOC, RPA2, RPL3, RPLP1, RPLP2, RPS27A, SAR1B, SCARB2, SKIL, SLC25A1, SLC25A6, SOD2, SPTAN1, SQRDL, SSBP1, SURF4, SUSD5, TAGLN2, TGFBI, TNPO1, TPI1, TRAM1, TUBA1A, TUBA1C, TUBB, TXN, TXNDC5, VIM, YWHAQ and YWHAZ, but is not limited thereto.

In addition, when the fibroblasts are derived from heart tissue, the extracellular matrix may include at least one of ACTN1, ACTM4, ANXA2, ANXA4, ANXA5, ANXA6, CAV1, COL1A1, COL1A2, COL3A1, COL5A2, COL6A1, COL6A2, COL6A3, FLNC, GPNMB, GSTM3, LMNA, LUM, MYH9, PLEC, PRDX1, PRDX4, PRELP, PTRF, THY1, VAT1, 4F2, 5HT3A, A5YM53, ACTG, ACTS, ADA30, ADT1, ALBU, ALDR, AMPN, ANAG, ANXA1, ANXA4, APOD, ARI4B, ASH1L, ASPG, AT1A1, AT1B3, AT2A2, ATPB, ATS9, B2MG, B2ZZ86, B7ZLE5, B8XPJ8, BASI, BGH3, BST1, CA2D1, CALR, CALX, CATA, CATD, CAV2, CAZA1, CBPZ, CC106, CD44, CD47, CD59, CD63, CD81, CD9, CHM4B, CLH1, COCA1, COL2A1, COL4A1, COL4A2, COL5A3, COPA, COPB, COX2, CSPG2, CTNA1, D3DTH7, D3DTX7, DAF, DERM, DPP4, EF1A1, EF2, EHD1, EHD2, EMIL1, ERD22, ERGI1, ERLN2, FA5, FBLN2, FBN1, FBN2, FBN3, FRAS1, FRIH, FRIL, G3P, GBB1, GBLP, GDN, GLCM, GNS, GPDM, GRP78, H2A2A, H31, H4, H6VRG3, H9NL19, HBB, HEXB, HM13, HNRH1, HNRPC, HSP7C, HTRA1, 16L9E7, IF16, IF4A2, IF4A3, IQGA1, ITA2, ITB1, ITB3, JAG2, K1C10, K1C14, K1C9, K22E, KPYM, LAMC1, LAMP1, LAMP2, LEMD2, LRP1, LRP1B, LRP2, LTBP3, MAGE1, MAST1, MBD2, MDHM, MFGM, MGST3, MRC2, MT1L, MT3, MYADM, MYH10, MYH9, MYOF, NB5R3, NEP, NICA, NID2, NPC1, NPM, NPTN, NU5M, OST48, PCYOX, PDIA1, PDIA4, PFKAL, PGBM, PGFRB, PGH1, PGH2, PGRC1, PGS1, PGS2, PLMN, PPIB, PSA7, PSB6, Q0VAS7, Q4KMR2, Q60FE6, Q6IPH7, Q6NZX3, Q8IWP6, Q9BUM6, RAB1B, RINI, RL18, RL3, RL40, RPN1, RS11, RS3, RS9, SAM50, SCRB2, SEC62, SEPR, SERA, SODM, SPCS2, SQRD, SSPO, SSRA, STAB1, STOM, SVEP1, SYPL1, TBA1A, TBA1B, TBB5, TBB6, TBL1R, TENA, TGM2, TITIN, TM201, TMEDA, TMM43, TPBG, TPIS, TRPV2, TRXR1, TSP1, U3KQK0, UBP7, USH2A, VAS1, VDAC1, VDAC2, VDAC3, VIME, VINC, VTNC, WFKN2 and WNT5A, but is not limited thereto.

Furthermore, the extracellular matrix according to an exemplary embodiment of the present invention may commonly include ACTN1, ACTN4, ANXA2, ANXA4, ANXA5, ANXA6, CAV1, COL1A1, COL1A2, COL3A1, COL5A2, COL6A1, COL6A2, COL6A3, FLNC, GPNMB, GSTM3, LMNA, LUM, MYH9, PLEC, PRDX1, PRDX4, PRELP, PTRF, THY1 and VAT1 regardless of the origin of the fibroblasts. Accordingly, the extracellular matrix according to an exemplary embodiment of the present invention may include at least one of ACTN1, ACTN4, ANXA2, ANXA4, ANXA5, ANXA6, CAV1, COL1A1, COL1A2, COL3A1, COL5A2, COL6A1, COL6A2, COL6A3, FLNC, GPNMB, GSTM3, LMNA, LUM, MYH9, PLEC, PRDX1, PRDX4, PRELP, PTRF, THY1 and VAT1.

At this time, since ACTN1 and ACTN4 may express actinin, the extracellular matrix according to an exemplary embodiment of the present invention may include actinin.

In addition, since COL1A1, COL1A2, COL3A1, COL5A2, COL6A1, COL6A2, and COL6A3 may express collagen, the extracellular matrix according to an exemplary embodiment of the present invention may include collagen.

In addition, since FLNC may express an actin-binding-like protein (filamin-C), the extracellular matrix according to an exemplary embodiment of the present invention may include the actin-binding-like protein.

Ultimately, the present invention may provide an extracellular matrix including a plurality of structural proteins, such as collagen, actinin, and an actin-binding-like protein (filamin-C).

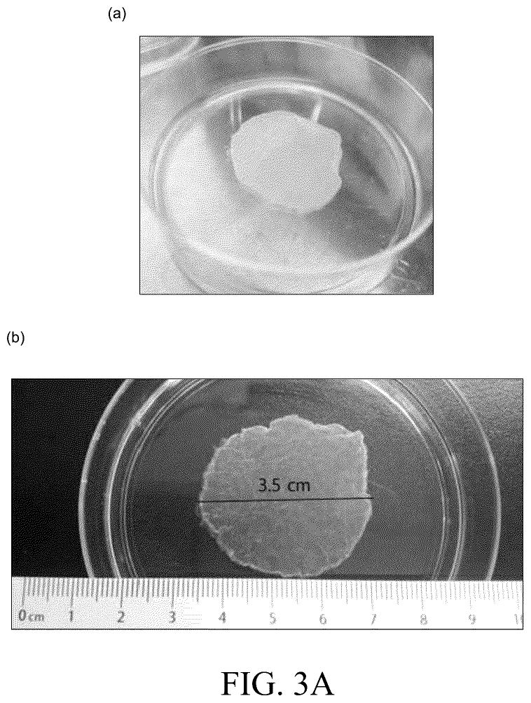

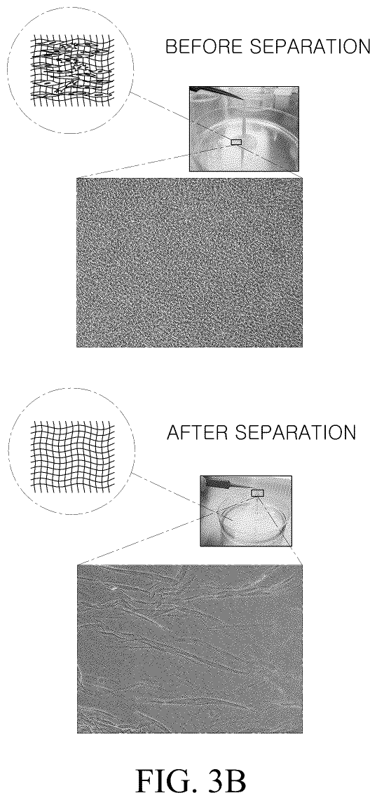

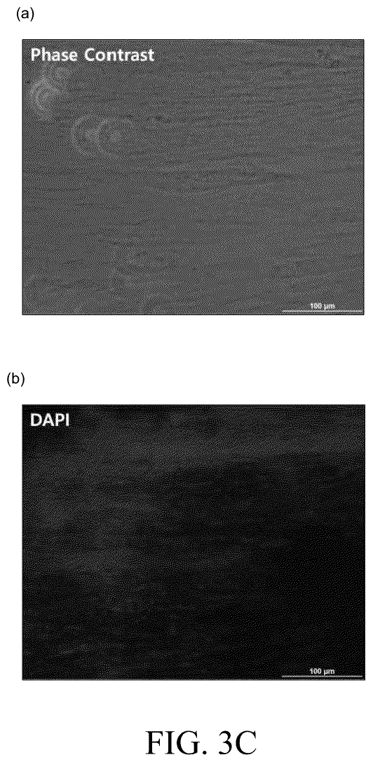

Referring to FIGS. 3A to 3C, observation images of the extracellular matrix according to an exemplary embodiment of the present invention produced through the above-described process are illustrated.

First, referring to FIG. 3A, the extracellular matrix according to an exemplary embodiment of the present invention may have predetermined area and thickness. At this time, the illustrated extracellular matrix may be an extracellular matrix including cells for observation of size and thickness, and accordingly, may be opaque.

More specifically, when the extracellular matrix of the present invention includes cells, the extracellular matrix has an area of about 1 cm² or more and a thickness of about 100 μm or more, and an average length may have a horizontal length of about 3.5 cm or more. In other words, in the extracellular matrix according to an exemplary embodiment of the present invention, cells are included in the extracellular matrix to be entangled into a skein or stacked without limitation (sandwich form), so that the extracellular matrix may have improved area and thickness compared to a conventional extracellular matrix, which has limited thickness (50 μm) and area (size). Accordingly, it is possible to provide a more improved three-dimensional culture environment for the formation of cell aggregates such as organoids and spheroids.

Meanwhile, when the extracellular matrix of the present invention does not contain cells, elasticity and compliance are lost and thus the extracellular matrix is stretched to have an average length of about 5 cm or more and a thickness of about 20 μm or more.

Furthermore, referring to FIG. 3B, the extracellular matrix according to an exemplary embodiment of the present invention in which the decellularization process has been performed does not include any cells and is structurally stable without rupture, and the extracellular matrix according to an exemplary embodiment of the present invention in which the decellularization process has been performed has the form of a thin membrane.

Furthermore, referring to FIG. 3C, the fluorescent staining results of the decellularized extracellular matrix according to an exemplary embodiment of the present invention are illustrated. At this time, the nucleus and the chromatin were contrastively identified using DAPI staining.

The decellularized extracellular matrix according to an exemplary embodiment of the present invention has no DAPI-stained cells or residues, and has a fiber form. Accordingly, when specific cells are cultured in the extracellular matrix according to an exemplary embodiment of the present invention, the cells may be easily differentiated into cell assemblages such as target spheroids and organoids without affecting other components. That is, the decellularized extracellular matrix according to an exemplary embodiment of the present invention may provide high tissue differentiation.

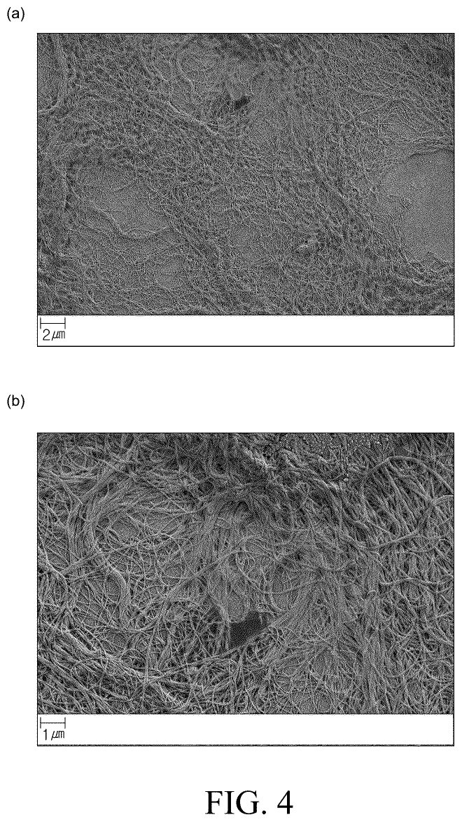

FIG. 4 illustrates electron microscopy image results for an extracellular matrix according to an exemplary embodiment of the present invention. At this time, the extracellular matrix according to an exemplary embodiment of the present invention is a decellularized extracellular matrix without containing cells.

Referring to (a) of FIG. 4, the extracellular matrix of the present invention is present in entangled nanofibers.

Referring to the more enlarged view of (b) of FIG. 4, the thickness of the nanofiber included in the extracellular matrix of the present invention is about 0.5 μm or less. In addition, it is shown that there is a fine gap between the entangled nanofibers, and accordingly, oxygen and the culture medium may move between the entangled nanofibers. That is, as the extracellular matrix according to an exemplary embodiment of the present invention includes the gap between the nanofibers, when the extracellular matrix includes cells, nutrients in the culture medium may be smoothly supplied up to the cells included the extracellular matrix.

Meanwhile, the shape of the extracellular matrix containing these nanofibers may mean the same structure as the extracellular matrix in vivo. More specifically, the extracellular matrix that has been commercially used in the art has been extracted from a specific tissue, dried and ground, and provided in a powder form. Accordingly, the conventional extracellular matrix did not preserve the nanofibers and was not realized. Furthermore, as the extracellular matrix extracted from the specific tissue may have limitations in a specific tissue sample to be supplied, the supply thereof may also be limited.

However, the extracellular matrix according to an exemplary embodiment of the present invention may be continuously supplied from a cell culture in vitro, rather than extracted from a specific tissue, and may have the same form as the extracellular matrix in vivo, thereby providing a more living body-like environment for cell culture. Accordingly, the extracellular matrix according to an exemplary embodiment of the present invention may provide an easier culture environment in three-dimensional cell culture, that is, an environment closer to the living body in an in vivo simulation of specific cells (cell aggregates such as spheroids and organoids).

Meanwhile, the extracellular matrix according to an exemplary embodiment of the present invention may be in a semisolid phase and may have elasticity and compliance.

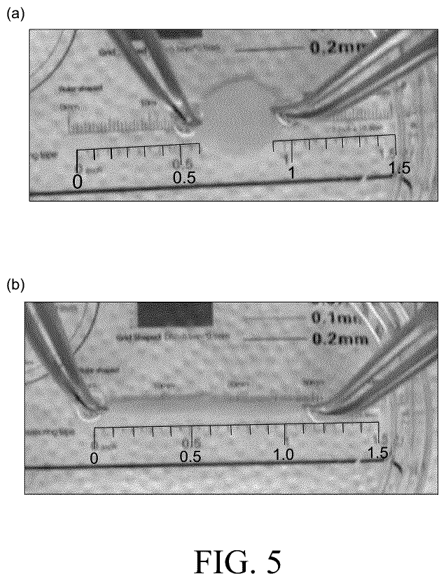

More specifically, referring to FIG. 5, observation images for phase changes of the extracellular matrix according to an exemplary embodiment of the present invention are illustrated.

The extracellular matrix according to an exemplary embodiment of the present invention has elasticity and compliance when including fibroblasts or target cells to be cultured, and may be recovered (restored) again after being stretched. Accordingly, the extracellular matrix of the present invention may be stretched from a length of about 0.5 cm to about 1 cm, which is twice larger than the length, so that the extracellular matrix of the present invention may have the compliance of about twice or more.

On the other hand, when the extracellular matrix of the present invention does not include cells, the elasticity and compliance may be lost. In other words, when the extracellular matrix of the present invention includes the cells, the elasticity and compliance are added, so that the cells cultured in the extracellular matrix may be cultured more safely and stably. Ultimately, the extracellular matrix of the present invention may safely protect cells from external shock (exposure).

According to the above results, the extracellular matrix according to an exemplary embodiment of the present invention may provide an optimized environment for differentiation and three-dimensional culture of cell aggregates such as target spheroids and organoids of specific cells.

### Verification of Use of Extracellular Matrix According to an Exemplary Embodiment of the Present Invention

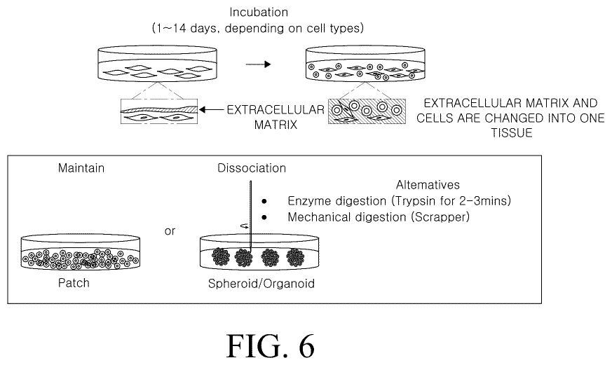

Hereinafter, the verification results for the extracellular matrix according to an exemplary embodiment of the present invention will be described with reference to FIGS. 6 to 9.

First, FIG. 6 is an exemplary diagram of a cell culture method of an extracellular matrix according to an exemplary embodiment of the present invention.

Unlike a conventional extracellular matrix, the extracellular matrix according to an exemplary embodiment of the present invention may not adhere to a plate, and may be elastically changed into a three-dimensional shape according to cells to be cultured.

More specifically, in conventional three-dimensional cell culture, specific cells are cultured on a plate coated with the extracellular matrix, so that the cells may be cultured laterally, similarly to a two-dimensional cell culture environment. Accordingly, the conventional extracellular matrix has a limitation in that in the case of floating cells and a three-dimensional culture environment, the extracellular matrix and the cells may not be combined, so that cell differentiation is not smooth.

However, the extracellular matrix according to an exemplary embodiment of the present invention is just present on the plate and is not attached, and when specific cells are added, the extracellular matrix may form a three-dimensional structure surrounding the cells. That is, the extracellular matrix according to an exemplary embodiment of the present invention may be a specialized extracellular matrix in three-dimensional cell culture. Furthermore, the extracellular matrix according to an exemplary embodiment of the present invention may surround the entire surface area of cells and culture the cells, thereby simulating an in vivo environment as closely as possible and protecting the cells from physical stimulation or stress as much as possible. Accordingly, the extracellular matrix according to an exemplary embodiment of the present invention may stably differentiate specific cells into desired target tissues.

Accordingly, referring to FIG. 6, the extracellular matrix of the present invention is applied on a medium containing cells to develop (differentiate) into cells and cell aggregates or tissues such as spheroids and organoids.

At this time, the cells and extracellular matrix that have developed into a single tissue may be continuously cultured in the form of a patch, and may be dissociated physically (mechanical digestion) or chemically (enzyme digestion) and cultured to be differentiated into spheroids or organoids.

Furthermore, the extracellular matrix according to an exemplary embodiment of the present invention may maintain the structural properties of a scaffold even when specific cells are differentiated and cultured and then decellularized. That is, as the extracellular matrix has predetermined thickness and size, the extracellular matrix is more robust than a conventional extracellular matrix and thus may be reused.

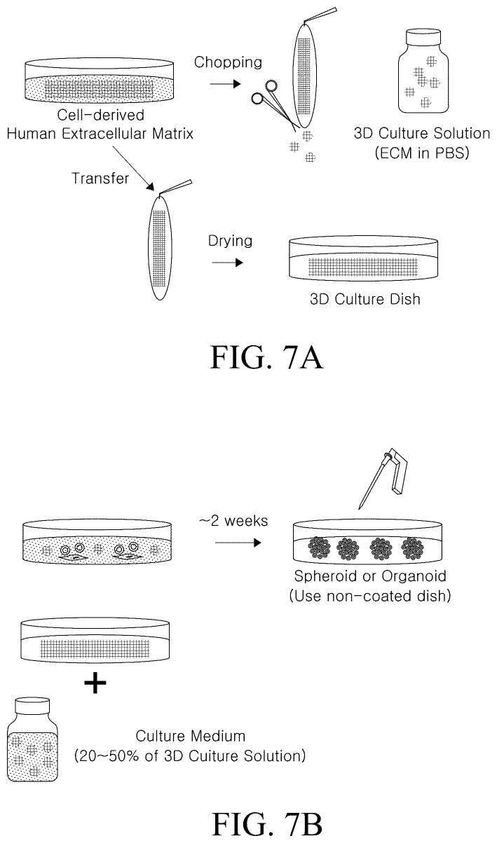

FIGS. 7A and 7B are exemplary diagrams of a composition for three-dimensional cell culture and a dish for three-dimensional cell culture including an extracellular matrix according to an exemplary embodiment of the present invention.

The extracellular matrix according to an exemplary embodiment of the present invention is provided in various forms for three-dimensional cell culture and thus may be used for cell culture.

First, referring to FIG. 7A, the extracellular matrix according to an exemplary embodiment of the present invention is chopped and stored in a suspension such as PBS or a culture medium to be provided as a composition for three-dimensional cell culture.

In addition, the extracellular matrix according to an exemplary embodiment of the present invention may be dried as obtained and then coated to a cell culture dish to be provided as a dish for three-dimensional cell culture.

Furthermore, referring to FIG. 7B, the composition for three-dimensional cell culture and the dish for three-dimensional cell culture according to an exemplary embodiment of the present invention may be used together in three-dimensional cell culture. More specifically, when cells to be cultured are added and cultured together with the composition for three-dimensional cell culture in the dish for three-dimensional cell culture according to an exemplary embodiment of the present invention, the cells may be surrounded and cultured by a large amount of extracellular matrix, thereby being more effectively differentiated into spheroids or organoids.

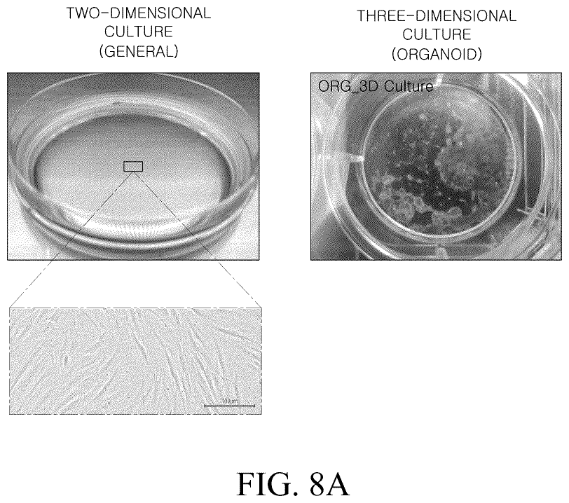

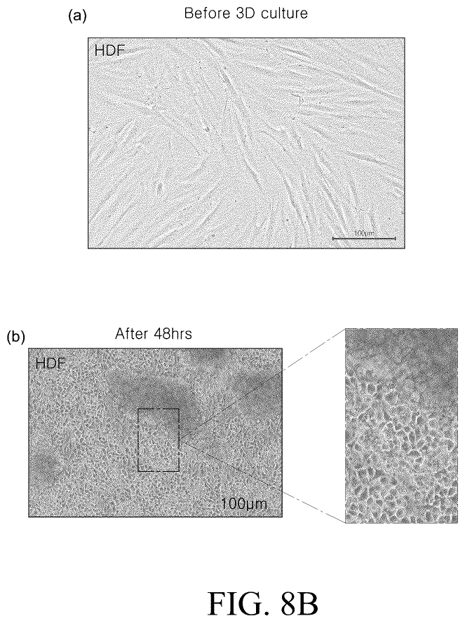

FIGS. 8A and 8B illustrate cell culture results in an extracellular matrix according to an exemplary embodiment of the present invention.

First, referring to FIG. 8A, when cells are cultured in a typical two-dimensional extracellular matrix, the cells do not grow into a plurality of cell layers, but mainly grow thinly spread out.

However, when the cells are cultured in the extracellular matrix according to an exemplary embodiment of the present invention, the cells form aggregates having a predetermined size and are cultured in suspension.

Furthermore, referring to FIG. 8B, a microscope image for FIG. 8B is shown, and cells cultured in the extracellular matrix according to an exemplary embodiment of the present invention have a spherical shape as shown in FIG. 8B, and form a plurality of cell layers.

That is, unlike general two-dimensional and conventional three-dimensional cultures, the extracellular matrix according to an exemplary embodiment of the present invention may completely surround and culture the cells to be cultured using elasticity and compliance.

Furthermore, since the extracellular matrix of the present invention has a predetermined thickness, the cells may be cultured by forming a plurality of layers in the extracellular matrix, thereby simulating a three-dimensional culture environment most similar to the in vivo environment.

In addition, the cells cultured in the extracellular matrix according to an exemplary embodiment of the present invention have cells having the same size and shape at the same time, so that the extracellular matrix of the present invention may culture uniformly identical cells without causing heterogeneous cells.

Although the exemplary embodiments of the present invention have been described in detail with reference to the accompanying drawings, the present invention is not limited thereto and may be embodied in many different forms without departing from the technical concept of the present invention. Therefore, the exemplary embodiments of the present invention are provided for illustrative purposes only but not intended to limit the technical concept of the present invention. The scope of the technical concept of the present invention is not limited thereto. Therefore, it should be appreciated that the aforementioned exemplary embodiments are illustrative in all aspects and are not restricted. The protective scope of the present invention should be construed on the basis of the appended claims, and all the technical ideas in the equivalent scope thereof should be construed as falling within the scope of the present invention.

1. A method for producing an extracellular matrix comprising the steps of:
culturing fibroblasts in a stimulation medium so as to form a fibroblast patch comprising the fibroblasts and an extracellular matrix;
treating the formed fibroblast patch with a proteinase;
freezing the proteinase-treated fibroblast patch;
thawing the frozen fibroblast patch so that the fibroblasts are decellularized from the fibroblast patch; and
obtaining the extracellular matrix from the decellularized fibroblast patch.

2. The method for producing the extracellular matrix of claim 1, wherein the stimulation medium includes 0.01 to 2 mM ascorbic acid.

3. The method for producing the extracellular matrix of claim 1, wherein the culturing step is performed for at least one period of 3 to 20 weeks.

4. The method for producing the extracellular matrix of claim 1, wherein the proteinase is 0.01 to 1% trypsin.

5. The method for producing the extracellular matrix of claim 1, wherein the freezing step is performed at a temperature of −10° C. or lower.

6. The method for producing the extracellular matrix of claim 1, wherein the thawing step is performed at room temperature for at least 2 hours.

7. The method for producing the extracellular matrix of claim 1, further comprising:
after the thawing step,
treating the thawed fibroblast patch with a proteinase.

8. The method for producing the extracellular matrix of claim 1, further comprising:
after the obtaining step,
freeze-drying the obtained extracellular matrix.

9. The method for producing the extracellular matrix of claim 1, wherein the extracellular matrix obtained in the obtaining step includes fibronectin.

10. The method for producing the extracellular matrix of claim 1, wherein the fibroblasts are derived from at least one of tendon, ligament, muscle, skin, periodontium, cornea, cartilage, bone, liver, blood vessels, heart, small intestine, large intestine, and intervertebral disc.

11. An extracellular matrix comprising collagen, actinin, and an actin-binding-like protein (filamin-C), and being present in the form of entangled nanofibers.

12. The extracellular matrix of claim 11, wherein the nanofibers have a thickness of about 0.5 μm or less.

13. The extracellular matrix of claim 11, wherein the extracellular matrix has elasticity and compliance when including cells, and
has no elasticity and compliance when not including the cells.

14. The extracellular matrix of claim 11, wherein the extracellular matrix allows the culture medium to be movable between the entangled nanofibers.

15. The extracellular matrix of claim 11, wherein the extracellular matrix comprises at least one of ACTN1, ACTN4, ANXA2, ANXA4, ANXA5, ANXA6, CAV1, COL1A1, COL1A2, COL3A1, COL5A2, COL6A1, COL6A2, COL6A3, FLNC, GPNMB, GSTM3, LMNA, LUM, MYH9, PLEC, PRDX1, PRDX4, PRELP, PTRF, THY1 and VAT1.

16. The extracellular matrix of claim 11, wherein the extracellular matrix is derived from fibroblasts in vitro.

17. The extracellular matrix of claim 11, wherein the extracellular matrix has the same structure as an extracellular matrix in vivo, and
is in a semisolid phase.

18. (canceled)

19. The extracellular matrix of claim 11, wherein the extracellular matrix is for three-dimensional cell culture.

20. A composition for three-dimensional cell culture, comprising the extracellular matrix of claim 11.

21-23. (canceled)
